(19) United States
(12) Patent Application Publication
(10) Pub. No.: US 2012/0190939 A1
(43) Pub. Date: Jul. 26, 2012

(54) METHOD FOR MONITORING THE MEDICAL CONDITION OF A PATIENT

(76) Inventor: Thomas Löser, Leipzig (DE)

(21) Appl. No.: 13/387,642

(22) PCT Filed: Jul. 29, 2010
(86) PCT No.: PCT/EP2010/061036

§ 371 Date: Apr. 3, 2012

(30) Foreign Application Priority Data
Jul. 29, 2009 (DE) 10 2009 035 250.3

(51) Int. Cl. A61B 5/00

(52) U.S. Cl. 600/301

(57) ABSTRACT

The invention relates to a method for monitoring the medical condition of a patient. The method comprises
(a) the specification of a first group of physiological and/or pathological parameters of the patient that are characteristic of the presence of a disturbance of health, wherein the parameters of the first group are to be determined in a continuous manner, and the specification of a second group of physiological and/or pathological parameters of the patient that are characteristic of the presence of a disturbance of health, wherein the parameters of the second group are to be determined in a discontinuous manner;
(b) the continuous recording of parameters of the first group;
(c) the continuous determination of the necessity to determine values of parameters of the second group on the basis of previously recorded values of parameters of the first group or on the basis of previously recorded values of parameters of the first group and the second group; and
(d) the recording of values of parameters of the second group, if in step (c) the necessity of the recording of values of parameters of the second group has been found, at any time at which such a necessity has been found.

The invention relates to a method for monitoring the medical condition of a patient as well as a system for carrying out this method.

In the public health system, in particular in hospitals, there are employed patient data management systems wherein the patient data are stored. In addition to the so called master data of the patient such as name, Christian name, and date of birth the patient data comprise the medical findings and initiated or already finished medical or caring measures. In the following such measures are also referred to as medical measures.

As a rule, the medical findings are laboratory results, for example in cardiac diseases relevant measured values such as the myoglobin value, the troponine I value, and the CKMB value; in respiratory diseases relevant measured values such as the blood gas analysis e.g., the pH value of the blood, the partial oxygen pressure of the blood, the partial pressure of carbon dioxide of the blood, and the oxygen saturation of the blood. Medical findings are further the result of testing sampling material derived from the patient for infections by pathogenic substances and/or organisms, for example germs such as viruses, bacteria, protists, fungi, parasites, viroids, or prions.

Inspection of the patient for the presence or absence of an infection with pathogenic substances and/or organism is of particular importance in the intensive care of patients, in particular with patients already suffering from a sepsis or are at risk of getting a sepsis during the treatment in the intensive care unit.

A sepsis is defined as “the entirety of life-threatening clinical symptoms and pathophysiological changes in response to the action of pathogenic germs and their products that invade the blood stream from an infective focus, activate the large biological cascade mechanisms and specific cell systems, and trigger formation and release of Immoral and cellular mediators” (Schuster, H. P., and Müller-Werdan, U.: Definition und Diagnose von Sepsis und Muitiorganversagen in Sepsis und MODS, Berlin 2005). If additionally an organ of the patient is acutely affected, then there is a severe sepsis.

Typical pathogens of a sepsis are Staphylococcus aureus, coagulase-negative staphylococci, enterococci, Escheria coli, Klebsiella spp., Enterbacter ssp., and Pseudomonas aeruginosa.

At the beginning of an intensive care, i.e. usually in the admission of a patient in the intensive care unit of a hospital, it is often not known whether the patient suffers from an infection or not. Well known is, however, which pathogens, in particular which germs, are already present in the intensive care unit. For this reason, in the course of a so called “calculated therapy” immediately at the beginning of the intensive care one or more medicaments are given to treat typical infections, in particular those infections caused by the germs known to be present in the unit. Such medicaments include, e.g. antibiotics, virostatics, and antimycotics, wherein the calculated therapy typically includes the dose of an antibiotic.

At the same time, at the beginning of the intensive care there is taken a sample from the patient that is tested for pathogenic germs. The test that is connected for example with the detection of antibodies or the genetic information of pathogens (e.g. species PCR) or the culturing of cell cultures is performed in specialized laboratories. The results of this test are typically available after three days. The results show what pathogenic germs and resistances are present in the patient. As soon as this is known the medical treatment is adapted to the actual condition of the patient.

The data stored in the patient data management systems are entered into the system via standardized interfaces (in hospitals HL7, in practices xDT) by the persons treating the patient as well as by the laboratory personnel and on request placed at the disposal of the treating person.

In particular, with multimorbid patients the number of data that have to be recorded for each individual patient by the patient data management system is extremely high.

However, due to the considerable medical advance at present with supposedly simple diseases a large number of data are recorded that often do not allow a proper analysis. Thus, it is not atypical that medical decisions are made on the basis of single data that are considered to be particularly conspicuous. Other data stored in the patient data management system that could support an other findings are ignored, so that due to rather arbitrarily selected data medical measures are taken that in complete analysis of all data could turn out to be false or insufficient.

A further serious disadvantage of known patient data management systems is the complicated control of the efficacy of the medical measures taken, in particular of the medication. In particular, in known systems it can be determined hardly and as a rule only very late what effects a taken medical measure has on the development of a patient's physical condition. It is further not atypical that the discontinuation of a medical measure, for example a medicament, is forgotten, namely because it is not evident at first sight that such an order does exist, which connection exits between said order and the condition of the patient, or which purpose the order should originally have.

Moreover, it is a widespread problem that indeed not all of the patient's data that should be recorded indeed are recorded. Nevertheless, it is often not evident to the responsible physicians and nurses that there are data missing.

On the other hand, in practice there are often recorded data that have no use for the diagnosis and therapy of a patient's disease. For example, according to rigid schemes there are requested laboratory values, wherein depending on the value regulations are made for example daily or weekly. Such measurements are time-consuming and costly and may put additional strain on the patient.

From US 2007/287931 A1 there is known a system and method for monitoring the medical condition of a patient comprising the specification of a group of physiological and/or pathologic parameters of the patient that are characteristic of the presence of an epileptic disease. Here, the parameters are determined in a continuous manner.

Object of the invention is to eliminate the disadvantages of the prior art. In particular, a method for monitoring the medical condition of a patient shall be indicated which enables a performance review of medical measures taken and thus a proper medical treatment that may be associated with a noticeable reduction of the period of treatment of a patient. Moreover, it shall be ensured that indeed all data of a patient that have to be recorded according to the specifications made are actually recorded after all, wherein the specifications are made such that certain data are recorded only if they are medically necessary. Further, a system for carrying out said method shall be indicated.

This object is solved by the features of claims 1 and 17. Suitable developments of the inventions result from the features of claims 2 to 16.

In accordance with the invention there is provided a method for monitoring the medical condition of a patient comprising
(a) the specification of a first group of physiological and/or pathological parameters of the patient that are characteristic of the presence of a disturbance of health, wherein the parameters of the first group are to be determined in a continuous manner, and the specification of a second group of physiological and/or pathological parameters of the patient that are characteristic of the presence of a disturbance of health, wherein the parameters of the second group are to be determined in a discontinuous manner;
(b) the continuous recording of values of parameters of the first group;
(c) the continuous determination of the necessity to determine values of parameters of the second group on the basis of previously recorded values of parameters of the first group or on the basis of previously recorded values of parameters of the first group and the second group; and
(d) the recording of values of parameters of the second group, if in step (c) the necessity of the recording of values of parameters of the second group has been found, at any time at which such a necessity has been found.

The method according to the invention has the advantage that values for the parameters of the second group are recorded only if it results from the parameters established so far that it would be necessary to determine these parameters. Thus, it is prevented that according to rigid schemes values for parameters are determined that are not needed for the diagnosis and therapy of the disturbance of health. Instead, values of said parameters are determined only if it results from the parameters established so far that said values are needed.

Preferably, the second group of the parameters includes such parameters that are determined in the existing medical practice in intervals of 24 hours or more, for example microbiological parameters, and/or medical laboratory parameters, in particular such parameters that are characteristic of the presence of an infection. For example, the second group of parameters comprises parameters obtained by means of blood analyses and/or results from bacteriological, virological, and mycological tests or tests for other germs such as for example prions.

Thus, according to the invention tests for pathogenic germs are performed only results from the already existing data that the test results could be useful for diagnosis and/or therapy. Likewise, it is no longer required to record laboratory values on every day. It is instead established from the existing values of the recorded parameters whether certain laboratory values are needed.

According to the invention, on the basis of the e existing data, i.e. the so far recorded values of parameters of the first and—if already present—the second group of parameters, it is continuously checked whether values for the parameters of the second group have to be recorded. The necessity of such a recording may for example be found in that for at least one parameter of the first group there is given a trigger value or a range of trigger values. If the continuously recorded value of said given parameter reaches the trigger value or the range of trigger values it is required to determine at least one value of at least one parameter of the second group. Here, it is given for which parameter or for which parameters of the second group values have to be determined if the trigger value or a range of trigger values of the given first parameter is reached. Here, various first parameters may be linked with the same parameter(s) of the second group, i.e. if only one of the continuously recorded values of parameters of the first group reaches a trigger value or the range of trigger values then the necessity for the recording of a value of a parameter or of values of different parameters of the second group is a fact. Further, parameters of the first group may be linked such that trigger values or ranges of trigger values for two or more of these parameters must be reached before it is certainly necessary that values for parameters of the second group have to be determined.

Alternatively or additionally, the determination of the necessary parameters of the second group may be made by the following method:
(c1) selection of at least three parameters of the first or second group, wherein at least one of the three parameters is a parameter of the first group,
(c2) formation of a multidimensional feature space, wherein each of the chosen parameters forms a dimension of the feature space and the time forms a further dimension of the feature space;
(c3) determination of at leas a first space range and at least a second space range within the feature space, wherein
   in the first space range each of the chosen parameters and/or the time have values that have been given as target values;
   in the second space range at least one of the parameters and/or the time have values that have been given as unwanted values;
(c4) continuous classification of the recorded values for the parameters chosen in step (c1) into the feature space to form measuring points in the feature space; and
(c5) determination of the necessity for recording parameters of the second group as soon as a measuring point is within the second space range.

If a measuring point is within the second space range values for a given parameter of the second group or for several given parameters of the second group are determined depending on what specifications have been made for the case that a measuring point is within a second space range. After the values of the parameters of the second group have been recorded a new second space range may be defined which considers the values of the first and second group recorded so far. Further, in the definition of the second space range also the medical measures taken so far, for example the administration of a medicament, may be considered.

It may be pointed out to the determined necessity for determining values of one or more parameters of the second group by means of an audible or optical alert on a display device, for example the display of a client computer of the patient data management system,

It may be found in carrying out the method according to the invention in step (c) that a parameter which initially has been assigned to the second group must be continuously determined. Said parameter may then be transferred from the second into the first group. For example, a parameter of the second group can automatically be transferred into the first group if it is found in step (c) that the determinations of this parameter had to be done in two or more sequential intervals of less than 24 hours.

In addition, the method according to the invention may comprise one or more of the following steps:
(e) recording the taken medical measures for the patient, wherein for each taken medical measure at least the time-related beginning of the medical measure is recorded and the medical measure is assigned to at least one of the recorded parameters of the first group that shall be affected with the medical measure:
(f) assigning the taken medical measure to a given category, wherein each category comprises a large number of potential medical measures;
(g) assigning the category to which the medical measure in step (c) has been assigned to one of the recorded parameters of the first group; and
(h) commonly and time-dependently representing (i) the category, (ii) the parameter of first group assigned in step (g), and (iii) at least one parameter of the second group.

By the common and time-dependent representation of (i) the category, (ii) the assigned parameter of the first group, and (iii) at least one parameter of the second group the physician can immediately recognize the connection between the condition of the patient, values of parameters characteristic of said condition, and the medical measures taken. For example, the physician can immediately recognize whether the patient suffers from an infection that has caused or will cause a sepsis. In this case, the second parameter is a microbiological parameter. Preferably, the common and time-dependent representation in step (h) comprises the representation of at least two parameters of the second group, wherein one of the two parameters is a microbiological parameter and the other of the two parameters is a laboratory-medical parameter.

The assignment takes place in the steps e) and (d) to one parameter of the first group each since only these parameters are recorded in a continuous manner. By a parameter of the first group it can also be understood a parameter that initially has been assigned to the second group, but was then transferred from the second group into the first group because it has been found in step (c) that the parameter must be determined in a continuous manner.

In one embodiment of the invention an optical or/and audible alert can be triggered if in the recording of values of parameters of the second group a given pathogenic germ and/or a resistance of a found germ to a drug that shall be prescribed for the patient is found.

Preferably, the taken medical measure is the administration of a drug, in particular the administration of a medicament.

It is preferred to set up the categories on the basis of given features that are inherent in a part of the potential medical measures only. Such features are for example the active ingredient of the drug, the group of the active ingredients of said drug, and/or the intended pharmacological effect of said drug.

For each medical measure that has to be taken for a certain patient the beginning of the medical measure is recorded. However, it is more suitable to record also further data about the medical measure in step (e). Such further data may be selected from the group comprising for example the intended end of the taken medical measure, the real end of the taken medical measure, the name of the prescribed drug, the pharmacological parameters such as the form of administration, the dose, the frequency of the dose per day, and the time-related distribution of the dosage during the day and the change of the dosage during an intended period of time as well as combinations of these data.

In a preferred embodiment the name of the prescribed drug is recorded wherein by means of the name of the prescribed drug the category of the taken medical measure is determined with recourse to a database wherein the name of the drug is linked with at least one feature of active ingredient, group of active ingredients, or intended pharmacological effect. For example, the indication of the name “Nebilet” or the name “Lobivon” can be linked with the active ingredient “Nebivolol” the group of active ingredients “β1-adrenoreceptor blockers”, and the pharmacological effect “lowering of the pulse rate and blood pressure”.

Since as a rule, it is not interesting for the success of a medical measure how the drug is called but only its active ingredient or group of active ingredients is of importance to such a success the creation of the category can be made for example in accordance with the feature “active ingredient“. That is, in step (h) in addition to the assigned parameter of the first group for example the category “β1-adrenoreceptor blockers” would be indicated that has been created on the basis of the feature “group of active ingredients”. The physician immediately recognizes in step (h) that a β1-adrenoreceptor blocker has been prescribed; which β1-adrenoreceptor blocker this actually is in the concrete is unimportant in this context. Of course, the physician can call up details of the prescribed medical measures that have been recorded in step (e) if he considers it necessary.

Preferably, the assignment of a medical measure to a category in step (e) is carried out automatically, i.e. the physician himself must not make the assignment of the measure taken by himself. It is rather determined automatically by means of the taken measure to which category said measure is assigned.

In one embodiment of the invention the parameter of the first group to which the category in step (g) is assigned is the same parameter as the parameter of the first group to which the medical measure in step (e) has been assigned. Alternatively or additionally, the parameter to which the category in step (g) is assigned is another parameter than the parameter to which the medical measure in step (e) has been assigned. The latter embodiment can be of advantage in particular in view of the broad-spectrum of a large number of drugs. For example, if a physician prescribes a β1-adrenoreceptor blocker to achieve a lowering of the blood pressure this may have effects on the patient's hepatic function. In this case, it may be advisable to link the category “β1-adrenoreceptor blocker” not only with the measured values of the blood pressure (a parameter of the first group) but also with parameters of the first group that are connected with the patient's hepatic function, for example with measured values of the glutamic-pyruvic transaminase (GPT), if said value has to be determined in a continuous manner.

The parameter(s) of the second group represented according to step (h) may be given by the physician or are automatically determined in that certain second parameters are assigned to certain first parameters, i.e. the displayed second parameter(s) may be linked to the parameters of the first group chosen in step (c) and/or (g) so that the assignment of a parameter of the first group in step (e) and/or (g) gives the parameter(s) of the second group that shall be displayed in step (h).

The parameters of the first and second group displayed in step (h) are commonly referred to in the following also as chosen parameters.

It is preferred that in step (h) the category and the chosen parameters of the first and second group are represented in a diagram the abscissa of which forms the time wherein in the diagram there are represented measured values of the chosen parameters and the category of the taken measure. Suitably, in this case the category is shown in the diagram as a bar running parallel to the abscissa wherein in particular the beginning of the measure is represented as the start of the bar in the diagram. At the end of the measure the presentation of the category, for example of the bar, in the diagram ends. In a further embodiment of the invention the category of the taken measure is represented as color-coded bar wherein the color-coding is linked with the given feature.

The term “physiological and/or pathological parameter” means a value that (a) indicates the measured value of a measured physiological characteristic of a patient or an indicator (e.g. the oxygen partial pressure in a blood analysis, respiratory rate, body temperature, CKMB concentration, germ load); and (b) can comprise a classification of a physiological condition of a patient (e.g. yellow coloration of the facial skin; presence of a certain germ; existence of a resistance).

A parameter is assigned to the first group if it is recorded in a continuous manner. Continuously recording means the uninterrupted recording of values of the parameter or the record of values of the parameter in intervals of 24 h or less.

A parameter is assigned to the second group if it is recorded in a discontinuous manner. Discontinuous recording means the single recording of values of the parameter; the recording of values of the parameter in regular or irregular intervals of more than 24 hours; or the complete absence of the recording of values for the parameter if it does not result in step (c) at any time of the method according to the invention that the recording of values for said parameter is necessary.

If it results in step (c) of the method according to the invention that a value initially having been assigned to the second group has to be recorded in intervals of 24 hours or less so this parameter is classified from the second group into the first group. Such a necessity may for example exist if a parameter initially having been assigned to the second group had been determined in two or more sequential intervals of less than 24 hours. For that, the method according to the invention can comprise recording the intervals between two sequential determinations of the parameters assigned to the second group and checking whether two or more sequential intervals between recordings of the same parameters are 24 hours or less. The number of sequential intervals that are 24 hours or less required for this should be an integer of greater than or equal to 2.

If a parameter is transferred from the second group into the first group an alert can be triggered and/or the category in step (e) can be assigned to said parameter which now is a parameter of the first group.

The term “indicator” herein relates to compounds or elements that—depending on their type—are produced in biological systems or are introduced in biological systems and the presence or concentration of which in a certain organ) is a characteristic for a biological process or a biological condition. For example, such compounds and elements comprise those produced by tumor cells, induced by a tumor in other body cells, and/or changed by a tumor as tumor-specific substances in their concentration. Such indicators are for example macromolecules, e.g. proteins, or trace elements. Such compounds and elements further comprise bone markers that are characteristic of osteoclasis processes such as osteoporosis or enzymes that are important for the assessment of the function of organs.

The term “value” or “measured value” herein relates (a) to all numerical values in the medical field for a physiological parameter that result (e.g. an oxygen partial pressure in a blood gas analysis of 81.2 mmHg, a respiratory rate of 15 breathes per minute, a body temperature of 36.8° C., CKMB concentration of 152 ng/ml; germ load), or (b) to classification results of a physiological condition of a patient (e.g. yellow coloration of the facial skin: no, wherein to classifications by means of statements such as “no” a numerical value, for example “0” should be assigned).

Preferably, at least the values of one of the physiological parameters that are characteristic of the presence of a disturbance of health are determined using an indicator.

Here, physiological parameters should be selected that due to medical findings are believed to be connected with a certain disturbance of health, for example a respiratory insufficiency. For determining the medical risk of a respiratory insufficiency there are employed for example physiological parameters that are obtained by means of a blood gas analysis. The physiological parameters obtained by means of the blood gas analysis can comprise the pH value of the blood, the blood's oxygen partial pressure, the blood's partial pressure of carbon dioxide, and the oxygen saturation of blood. Further physiological parameters that can be used to assess the medical risk of a respiratory insufficiency in the method according to the invention comprise in addition to the values resulting from the blood gas analysis the respiratory rate (AF) of the patient as well as the age and sex of the patient.

The number of parameters of the first group, the values of which are recorded in step (a), should be at least 2. The number of parameters of the second group, the values of which are recorded in step (d), should be at least 1, preferably at least 2.

The term “medical measure” comprises every medical or caring measure that is taken to improve the physical condition of the patient, for example to lead one or more of the recorded physiological parameters into a range that is typical of healthy persons.

The taken medical measures can also be recorded continuously, i.e. every further measure, every change of an existing measure (e.g. of the dosage of a medicament) and/or the real end of a measure are recorded.

In a further embodiment of the method according to the invention an additional step may be provided wherein the completeness of the data is examined. The examination of the completeness of the data may be continuous. Alternatively, it is carried out at expiry of a given period of time, for example every four, eight, sixteen, and/or twenty-four hours. Said time intervals may correspond to the duration of a shift or a working day of the hospital.

The data are considered complete if they comply with the medical guidelines, in particular with the taken medical measures as well as the physiological parameters and other information that have to be recorded in accordance to the guidelines of the hospital in general and the guidelines of the physician in particular.

The data that have to be recorded in accordance to the medical guidelines are in the following also referred to as “expected data”, since their recording in accordance to the medical guidelines is expected. Single expected data are referred to as “expected indication”.

In order to examine the completeness of the data the actually recorded data are compared with data that have to be recorded in accordance to the medical guidelines. In that occasion, the recorded data are assigned to the expected data. For example, when the recording of the oxygen partial pressure is expected, then the value actually measured and recorded for the oxygen partial pressure is assigned to the expected value. Each expected indication can be linked with one or more specified times from which it is evident when a value is expected for the expected indication.

The comparison of the recorded data with the expected data either results in that (i) all expected data also correspond to recorded data, i.e., that all expected data have also been recorded; that (ii) only a part of the expected data corresponds also to recorded data, i.e., that only a part of the expected data have been recorded, while another part of the expected data has not been recorded; or that (iii) none of the expected data correspond to recorded data, that none of the expected data have been recorded. In the cases (ii) and (iii) preferably a warning is emitted after a given time interval. For that, the fields in the screen presentation can be provided for example with a color-coding. Preferably, the color of the border and/or inscription of the field is changed if one of the cases (ii) or (iii) arises. In a preferred embodiment a warning is only emitted at the given time intervals, namely every four, eight, sixteen, and/or twenty four hours, in order to avoid a permanent warning each for another expected indication. In other words, when an examination of the completeness of the recorded data is set after four hours, then beforehand no warnings are emitted, even if the specification has not been met at an earlier point of time, for example already one hour before the expiry of the time interval of four hours.

When the monitoring sheet that is used by the respective hospital is deposited in the system for monitoring the medical condition of a large number of patients then the warning happens by color-coding of the respective areas of the monitoring sheet shown on the screen wherein the expected indication for which no data have been recorded are present. In this case, no warnings are emitted for the individual fields in which an expected indication remained without recorded data. In these areas expected indications are summarized in accordance to given criteria. Such areas are for example “perfusion”, “infusion”, and “decubitus” each comprising several expected data.

By selection of such an area a documentation sheet can be called up on the screen wherein then the expected indication is characterized for example by color-coding for which no data have been recorded.

Further, in accordance to the invention a system for monitoring the medical condition of a large number of patients according to the method of the present invention is provided. The system comprises a processor, a memory, an input device, and a display device, wherein
the input device enables the user to indicate the taken medical measures for each patient;
in the memory the continuously recorded values of parameters of the first and the second group of the large number of patients and the taken medical measures for each of these patients are stored;
the processor determines the necessity whether values of parameters of the second group have to be determined (step (c)) and, if intended, assigns the taken medical measure to a given category and assigns the category to one of the recorded parameters of the first group being characteristic of the presence of a disturbance of health; and
the display device commonly and time-dependently represents the category and the chosen parameters.

Furthermore, in the memory there can be deposited databases for the categories and given features to form the categories.

It is further possible that the system comprises a device for the examination of the completeness of the data.

The system may be a computer-implemented system.

In the following, the invention is explained in more detail with respect to drawings.

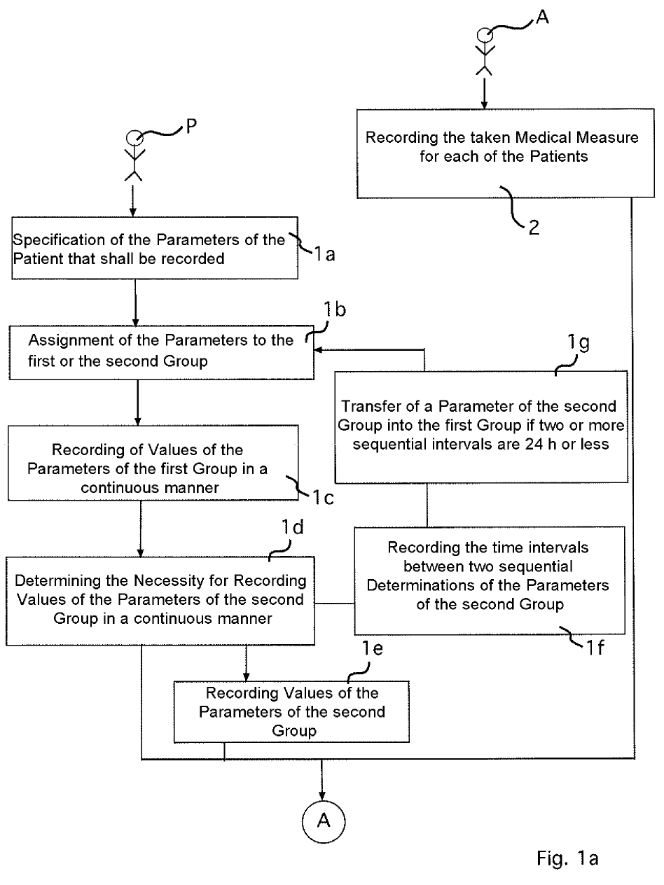

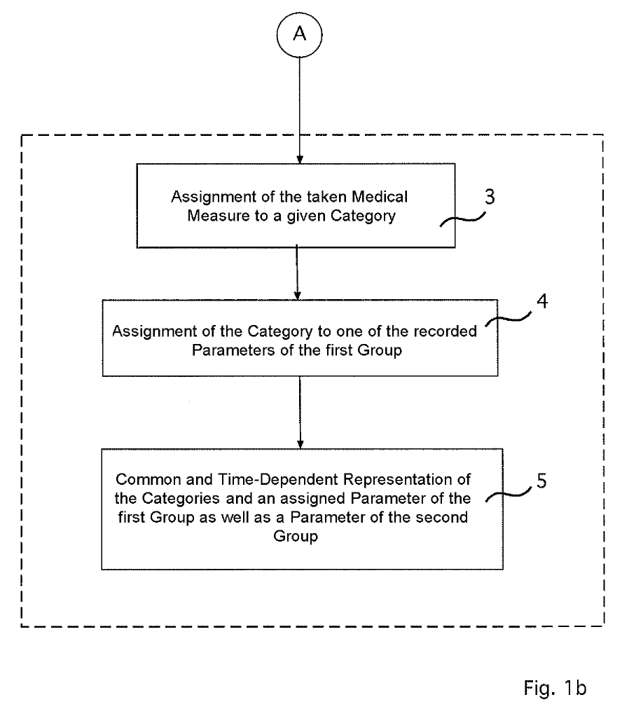

FIG. 1 shows a schematic representation of a first embodiment of the method according to the invention;

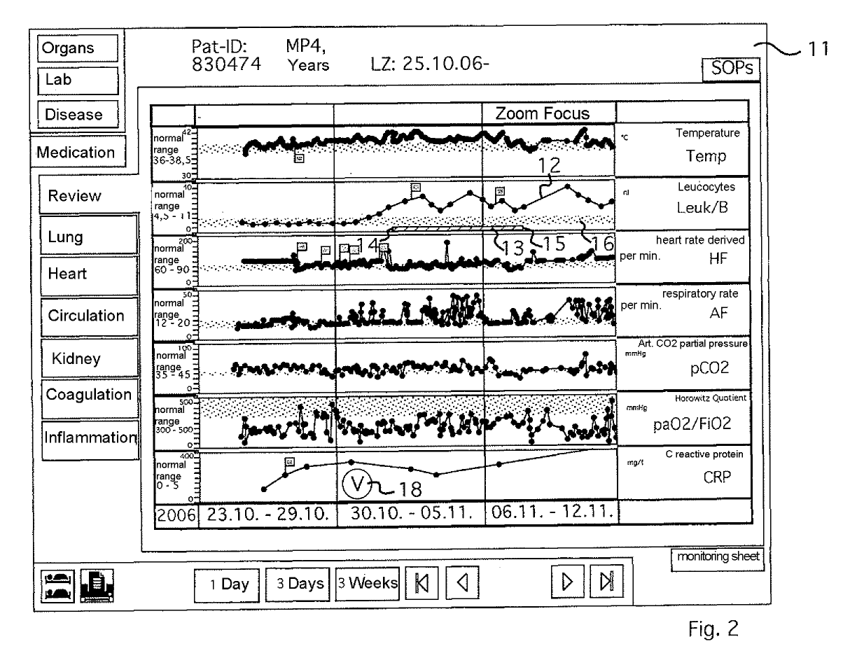

FIG. 2 shows a schematic representation of a screen image of the display device according to the invention;

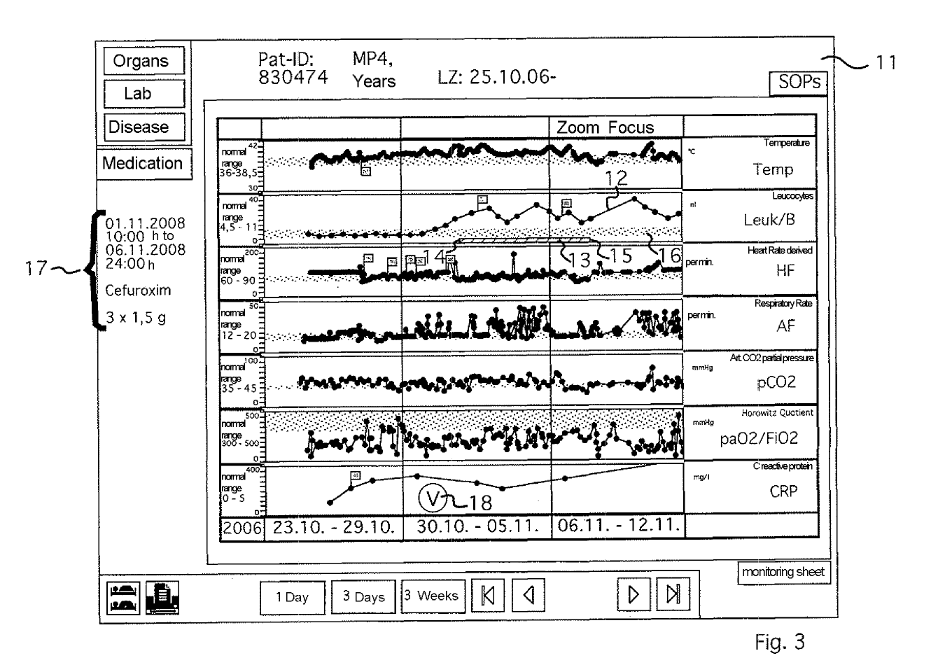

FIG. 3 shows a further schematic representation of a screen image of the display device according to the invention;

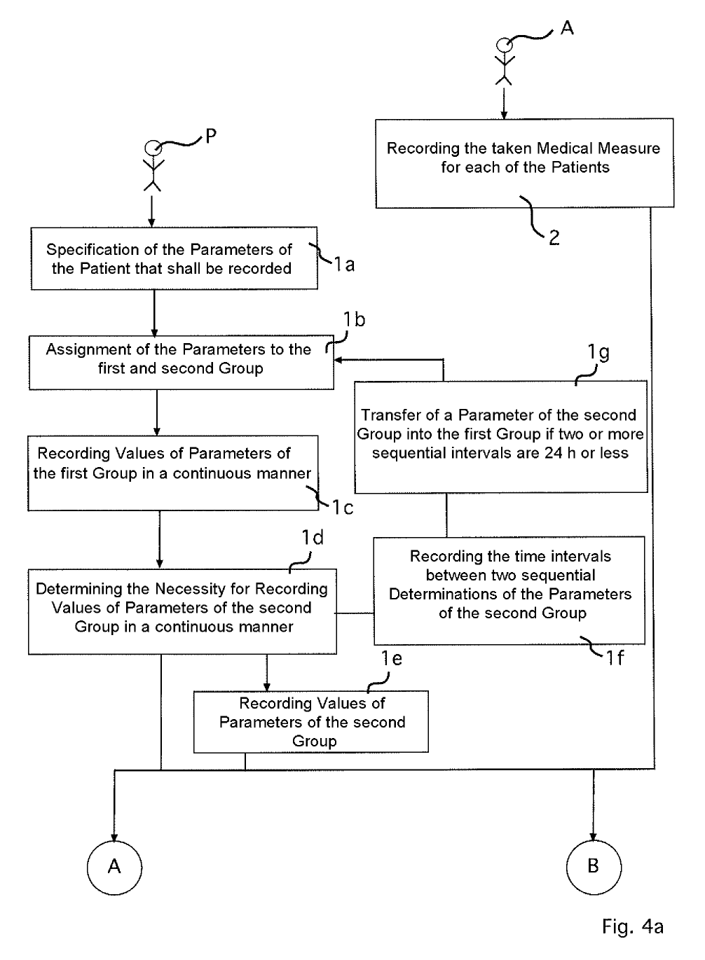

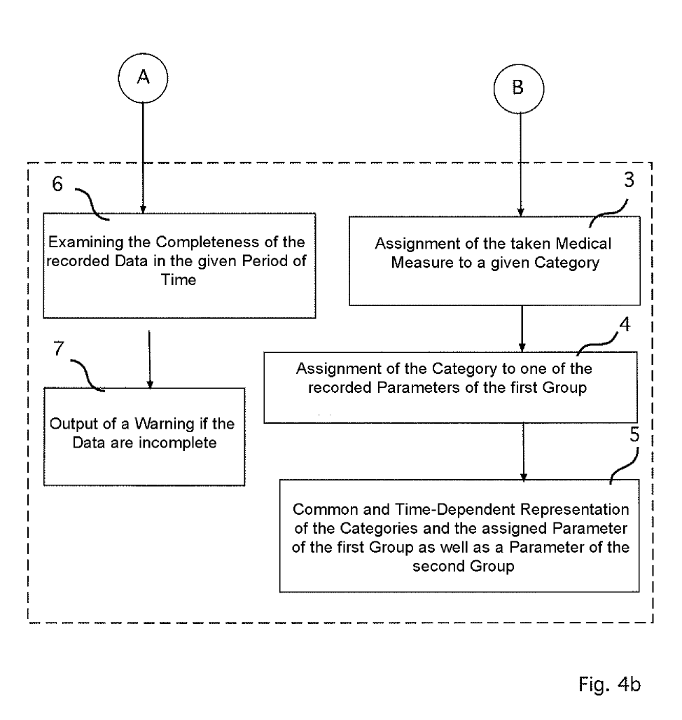

FIG. 4 shows a schematic representation of a second embodiment of the method according to the invention;

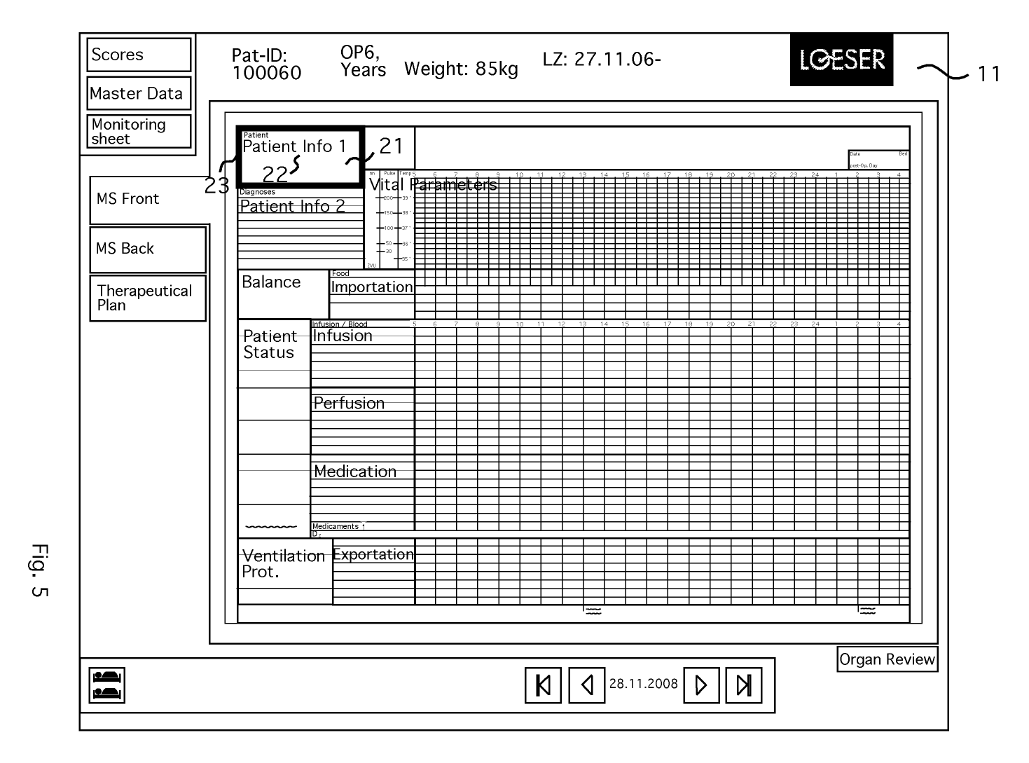

FIG. 5 shows a schematic representation of a screen image of the display device according to the invention, and

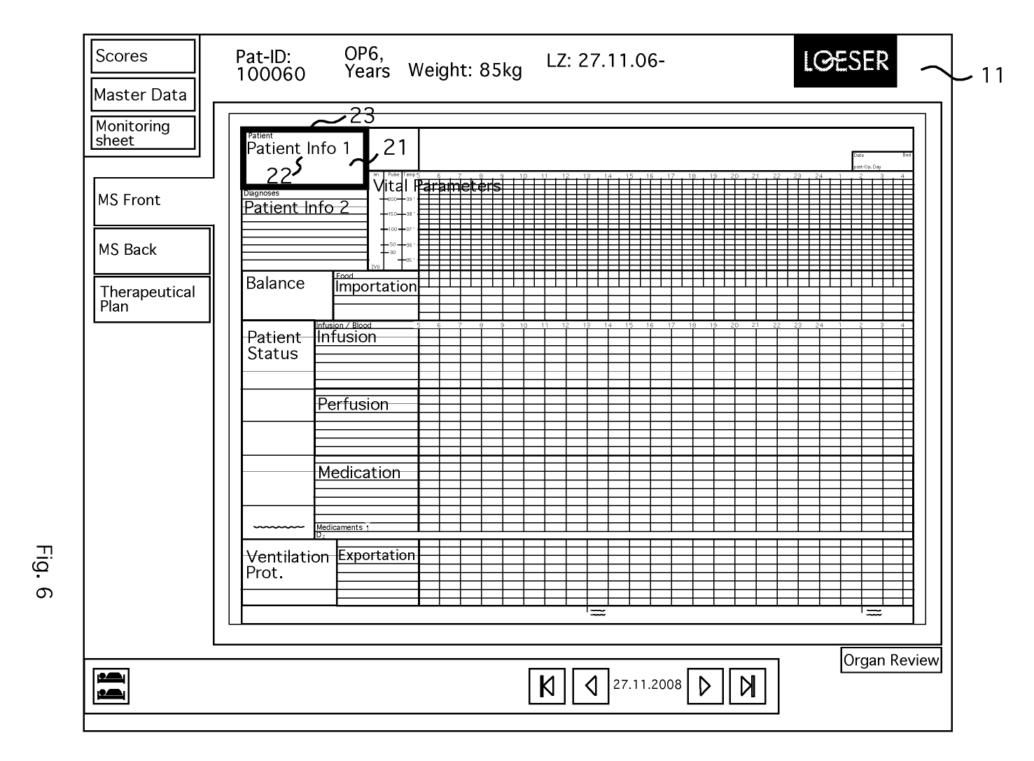

FIG. 6 shows a further schematic representation of a screen image of the display device according to the invention.

According to FIG. 1 in a first embodiment of the invention there are determined die physiological and/or pathological parameters of the patient P that shall be recorded (1a). Here, the parameters are assigned to the first group or the second group (1b). This assignment may be done by the physician. Alternatively, the assignment may already be stored in the system. For the parameters of the first group values are recorded in a continuous manner (1c). At the same time, it is continuously checked whether it is necessary to determine values of parameters of the second group (1d). This check is done on the basis of previously recorded values of parameters of the first group or on the basis of previously recorded values of parameters of the first group and the second group. Whenever such a necessity is found values of parameters of the second group are recorded (1e). At the same time the time intervals between the sequential records of the parameter of the second group are determined (1f). If two or more sequential intervals with a parameter of the second group are 24 h or less so this parameter is automatically classified from the second group into the first group (1g). The required number of such sequential intervals of 24 h or less may be given by the system or the physician.

During the so-made record of values of parameters the physician A, for example a doctor or a nurse, now takes a medical measure in view of the condition of the patient P reflecting in the recorded parameters of the first and second group. This may be the prescription of a medicament.

The medical measure that has been taken by the physician for a certain patient is recorded by the system according to the invention, for example by means of an input device 2. In that occasion, the beginning of the taken medical measure, the intended end of the taken medical measure, the name of the prescribed medicament, the form of administration, the dose, the frequency of the dose per day, and the time-related distribution of the dosage during the day, and the change of the dosage during a given period of time are recorded. Moreover, the physician will assign the medical measure to at least one of the recorded parameters of the first group that is to be affected with the medical measure.

Changes of the measures occurring later in time as well as their real end are also recorded over the time.

Then, the taken medical measure is assigned to a given category 3. Then, the category is assigned to one of the recorded parameters of the first group, as a rule, to the parameter that is to be affected with the taken medical measure.

Finally, the category and the chosen parameters of the first and second group are represented commonly and time-dependently on the display device of the system according to the invention 5.

Screen images 11 of the display device are shown in FIGS. 2 and 3. There, a part of the recorded parameters is shown, namely of parameters of the first group and of parameters of the second group. The parameters of the first group shown are body temperature (Temp [° C.] of the patient, heart rate (HF [min⁻¹]), respiratory rate (AF [min⁻¹]), arterial partial pressure of carbon dioxide (pCO2 [mmHg]), and the Horowitz quotient (paCO2/FiO2 [mmHg]). The parameters of the second group are the white blood cell count (Leuk/B [nl]), concentration of the C-reactive protein (CRP [mg/l]) as well as the establishment of meningococci.

The parameter 12, i.e. white blood cell count, is represented graphically as a function of time forming the abscissa. The physiological parameter 12 is the assigned parameter (step d) in accordance to the present invention. Said physiological parameter is linked with a taken medical measure, as is shown by the bar 13. The bar 13 reveals with respect to the abscissa the start 14 and the end 15 of the taken medical measure. The medical measure is assigned to a particular category, as is evident from the hachures of the bar 13 standing for a particular color-coding. In addition, a further bar 16 is shown characterizing the desired range of the physiological parameter 12.

Moreover, the diagnosis of a viral infection is indicated by a symbol 13 that points physician to the detection of meningococci in a sample of the patient.

By the common and time-dependent representation of category and chosen parameters a physician is readily able to recognize that a medical measure was taken. On the basis of the coded representation of the category he can further recognize, what kind of medical measure has been taken. He is thus able to link the measured values of the chosen parameters immediately with the taken medical measure involving a considerable advantage in view of the therapeutic success and thus the duration of treatment.

When the physician wishes more information regarding the taken medical measure, then he can choose to view them by selection of the bar by means of the input device, as is shown in FIG. 3, reference mark 17. He can then recognize that the active ingredient “Cefuroxim” has been prescribed, namely from Nov. 1, 2008 (beginning of the medical measure until Nov. 6, 2008 (end of the medical measure), with a dose of 1.5 g three times a day.

In that occasion, the active ingredient “Cefuroxim” that is for example commercially sold under the name “Zinacef” has been assigned to the category “β-lactam antibiotics”. The aim of the medical measure was to control a bacterial inflammation. It is evident to the physician from the diagram for the white blood cell count as assigned physiological parameter 12 that the prescription of the active ingredient “Cefuroxim” had the following effect: The inflammation was successfully controlled for a time reflecting in the drop of the measured value.

By choosing symbol 18 the physician gets detailed information about the diagnosed meningococcus infection, for example the type of the meningococci, their number, and optionally of established resistances of the patient.

Of course there can be shown further taken medical measures and their chosen parameters simultaneously with the medical measure shown in FIG. 2 and FIG. 3 and its chosen parameters.

In FIG. 4 there is shown a second embodiment of the method according to the invention corresponding to the first embodiment (see FIG. 1) except that additionally an examination of the recorded data for completeness is carried out 6. When in this context the recorded data are proved to be incomplete a warning is emitted 7. In FIGS. 5 and 6 a screen image 11 is shown representing a monitoring sheet. The fields of the monitoring sheet are divided in areas 21 wherein several expected indications are summarized. The areas are provided with an inscription 22 and a color-coding. If the examination shows after the given period of time that the recorded data are incomplete then the color of the inscription 22 of the area 21 and the color of the border 23 of the area 21 are changing. In FIG. 5 the screen image 11 is shown prior to the examination 6, whereas in FIG. 6 the screen image 11 is shown after the examination 6. In FIG. 5 each of the areas 21 has a black inscription 22 and a black border 23. It can be seen in FIG. 6 that warnings have been emitted 7 that are represented by the white border 23 of the respective areas 21. Alternatively or additionally, also a white inscription 22 of the respective areas 21 would be possible.

Here, the screen image 11 corresponds—apart from the inscription and color-coding of the areas 21—to the monitoring sheet that is typically used by the hospital.

### LIST OF REFERENCE MARKS

P patient
A physician
1a specification of the parameters of the patient P that shall be recorded
1b assignment of the given parameters to the first group or the second group
1c continuous recording of values of parameters of the first group
1d continuous determination of the necessity for recording values of parameters of the second group
   1e discontinuous recording of values of parameters of the second group
   1f recording of the time intervals between records
   1g transfer of one parameter from the second group into the first group
   2 recording of medical measures
   3 assignment of the medical measures to a category
   4 assignment of the category to a recorded physiological parameter
   5 representation of category and assigned parameter
   11 screen image of a display device
   12 assigned physiological parameter
   13 medical measure taken
   14 beginning of the medical measure
   15 end of the medical measure
   16 desired range of the assigned parameter of the first group
   17 details about the medical measure taken
   18 identification of the diagnosis of a virus
   21 area
   22 inscription of the area
   23 border of the area

Having thus described the invention, it is now claimed:

1. A method for monitoring the medical condition of a patient comprising
(a) the specification of a first group of physiological and/or pathological parameters of the patient that are characteristic of the presence of a disturbance of health, wherein the parameters of the first group are to be determined in a continuous manner, and the specification of a second group of physiological and/or pathological parameters of the patient that are characteristic of the presence of a disturbance of health, wherein the parameters of the second group are to be determined in a discontinuous manner;
(b) the continuous recording of values of parameters of the first group;
(c) the continuous determination of the necessity to determine values of parameters of the second group on the basis of previously recorded values of parameters of the first group or on the basis of previously recorded values of parameters of the first group and the second group; and
(d) the recording of values of parameters of the second group, if in step (c) the necessity of the recording of values of parameters of the second group has been found, at any time at which such a necessity has been found.

2. The method according to claim 1, characterized in that the second group of the parameters comprises microbiological parameters and/or medical laboratory parameters characteristic of the presence of an infection.

3. The method according to claim 1, characterized in that the second group of parameters comprises parameters obtained by means of blood analyses.

4. The method according to claim 1, characterized in that the second group of parameters comprises the results of bacteriological, virological, and mycological tests.

5. The method according to claim 1, characterized in that for the determination of the necessity for determining parameters of the second group (step (c)) for at least one parameter of the first group there is given a trigger value or a range of trigger values at which, if achieved, at least one given value of the second group has to be determined.

6. The method according to claim 1, characterized in that the determination of the necessity for determining parameters of the second group (step (c)) comprises
(c1) selection of at least three parameters of the first or second group, wherein at least one of the three parameters is a parameter of the first group,
(c2) formation of a multidimensional feature space, wherein each of the chosen parameters forms a dimension of the feature space and the time forms a further dimension of the feature space;
(c3) determination of at least a first space range and at least a second space range within the feature space, wherein
in the first space range each of the chosen parameters and/or the time have values that have been given as target values;
in the second space range at least one of the parameters and/or the time have values that have been given as unwanted values;
(c4) continuous classification of the recorded values for the parameters chosen in step (c1) into the feature space to form measuring points in the feature space; and
(c5) determination of the necessity for recording parameters of the second group as soon as a measuring point is within the second space range.

7. The method according to claim 1, characterized in that it further comprises:
(e) recording the taken medical measures for the patient, wherein for each taken medical measure at least the time-related beginning of the medical measure is recorded and the medical measure is assigned to at least one of the recorded parameters of the first group that shall be affected with the medical measure;
(f) assigning the taken medical measure to a given category, wherein each category comprises a large number of potential medical measures;
(g) assigning the category to which the medical measure in step (c) has been assigned to one of the recorded parameters of the first group; and
(h) commonly and time-dependently representing (i) the category, (ii) the assigned parameter of the first group, and (iii) at least one parameter of the second group.

8. The method according to claim 7, characterized in that the common and time-dependent representation in step (h) comprises the representation of at least two parameters of the second group, wherein one of the two parameters is a microbiological parameter and the other of the two parameters is a laboratory-medical parameter.

9. The method claim 7, characterized in that an optical or/and audible alert is triggered if in the recording of values of parameters of the second group a given pathogenic germ and/or a resistance of a found germ to a drug that shall be prescribed for the patient is found.

10. The method according to claim 7, characterized in that by means of the name of the prescribed drug the category of the medical measure taken is determined with recourse to a database wherein the name of the drug is linked with at least one feature of active ingredient, group of active ingredients, or intended pharmacological effect.

11. The method according to claim 7, characterized in that in step (e) in addition to the beginning of the medical measure the intended and/or actual end of said medical measure is recorded.

12. The method according to claim 7, characterized in that in step (e) the category, the assigned parameter of the first group, and the at least one parameter of the second group are represented in a diagram the abscissa of which forms the time wherein in the diagram there are represented measured values of the assigned physiological parameter and the category of the taken measure.

13. The method according to claim 12, characterized in that the category of the taken measure is represented as color-coded bar wherein the color-coding is linked with the given feature.

14. The method according to claim 1, characterized in that it further comprises the examination of the completeness of the recorded data and the output of a warning if the recorded data are entirely or partially incomplete.

15. The method according to claim 1, characterized in that the time intervals between two sequential determinations of parameters assigned to the second group are recorded and that a parameter is transferred from the second group into the first group if a given number of sequential time intervals that are lower than a given unit of time is achieved.

16. The method according to claim 15, characterized in that the given number is an integer greater than or equal to two and the given unit of time is 24 hours or less.

17. A system for monitoring the medical condition of a plurality of patients according to the method of claim 1, comprising a processor, a memory, an input device, and an display device, wherein:
(a) the input device enables the user to indicate the taken medical measures for each patient;
(b) in the memory the recorded values of parameters of the first and the second group of the plurality of patients and the taken medical measures for each of these patients are stored;
(c) the processor determines the necessity whether values of parameters of the second group have to be determined (step (c)) and, if intended, assigns the taken medical measure to a given category and assigns the category to one of the recorded parameters of the first group being characteristic of the presence of a disturbance of health; and
(d) the display device commonly and time-dependently represents the category, the assigned parameter of the first group, and at least one parameter of the second group.
